(19) United States
(12) Patent Application Publication
(10) Pub. No.: US 2004/0096424 A1
(43) Pub. Date: May 20, 2004

(54) Modification of gene expression in transgenic plants

(76) Inventors: Wolf B. Frommer, Tubingen (DE); Wolf-Nicolas Fischer, Palo Alto, CA (US); Brigitte Hirner, Dresden (DE); Sylvie Lalonde, Kusterdingen (DE); Sakiko Okumoto, Tubingen-Pfrondorf (DE); Mechthild Tegeder, Pullman, WA (US); John Ward, Falcon Heights, MN (US); Andreas Weise, Freibing (DE)

Correspondence Address: Stephan A Pendorf, Pendorf & Cutliff, PO Box 20445, Tampa, FL, 33622-0445, US

(21) Appl. No.: 10/258,789

(22) PCT Filed: Apr. 24, 2001
(86) PCT No.: PCT/EP01/04585

(30) Foreign Application Priority Data
Apr. 28, 2000 (EP) 00109218.8

(51) Int. Cl. A61K 38/19

(52) U.S. Cl. 424/85.1

(57) ABSTRACT

The invention relates to a regulatory element for the modification of gene expression in transgenic plants, to nucleotide enhancer and repressor sequences derived from non-coding regions of a sucrose transporter gene, and to suitable promoters for use in a regulatory element for the modification of gene expression in transgenic plants.

The object of the invention is to provide means for manipulating expression of plant genes, e.g. to control transport, storage, and growth processes in plants.

This problem is solved by a regulatory element comprising a promoter and a nucleotide sequence, wherein said nucleotide sequence is a nucleotide enhancer and/or repressor sequence comprising a non-coding region of a sucrose transporter gene.

### BACKGROUND OF THE INVENTION

1. Field of the Invention

The present invention relates to a regulatory element for the modification of gene expression in transgenic plants, to nucleotide enhancer and repressor sequences derived from non-coding regions of a sucrose transporter gene, and to suitable promoters for use in a regulatory element for the modification of gene expression in transgenic plants. The invention further relates to vectors, host cells, plant cells, plants, seeds, flowers, and other parts of plants, comprising a regulatory element for the modification of gene expression in transgenic plants.

2. Description of the Related Art

Plants use a vascular system, the phloem, for long distance transport of assimilated carbon compounds from photosynthetically active or storage tissues and organs (source tissues/organs) to non-photosynthetic parts of the plant (sink tissues/organs), where these compounds are consumed. Main components of this transport system are connected cells, so-called sieve cells or sieve elements, and their companion cells. Sieve cells are enucleate in higher plants and are kept alive by the companion cells, which produce micro- and macromolecules and translocate these substances via plasmodesmata into the sieve elements. The sieve cells form the conduits for long-distance transport in plants. Therefore, substances synthesized in companion cells such as proteins, peptides, RNAs, and other products of enzymatic reactions can be distributed from source to sinks via sieve elements. Substances that are transported into companion cells or sieve elements also can be distributed from source to sink via sieve elements.

The disaccharide sucrose is the major transported form of carbohydrates in plants. It is synthesized in green leaves and is transported via the phloem to support growth of sink organs, such as roots, meristems and flowers, or to support storage, e.g. in tubers. There are three principal locations in plants where sucrose uptake transporters in the plasma membrane are thought to be crucial for long-distance transport. First, in most plants, sucrose must be actively transported into the phloem cells in a process called phloem loading. This loading process is supposed to create the driving force for long-distance transport. Second, sucrose transporters serve in re-uptake along the length of the phloem to prevent excessive losses during transport and to recharge the driving force continuously. Third, in sink tissues such as meristematic regions and storage organs such as tubers and fruits, sucrose transporters are responsible for sucrose uptake by sink cells.

Sucrose transport at each of these locations is mediated and regulated by a set of specific plasma membrane proteins, the sucrose transporters (SUTs). Sucrose transporters are further required for sucrose efflux from cells located near the phloem in source leaves, for sucrose efflux from the phloem in sink tissue, and for transport across the vacuolar membrane.

The first sucrose transporter, SUT1, was cloned by functional expression in yeast (Riesmeier et al. 1992, EMBO J. 11: 4705-4713). Related genes from plants have since been obtained using the sequence for SUT1, including three genes from tomato (Lycopersicon esculentum). LeSUT1 and its orthologs from other plants are hydrophobic proteins consisting of 12 membrane spanning domains and are located in the plasma membrane of cells mediating highly specific influx of sucrose using a proton-coupled mechanism. The use of transgenic plants specifically impaired in SUT1 expression has provided strong evidence that SUT1 function is required for phloem transport (Riesmeier et al. 1994, EMBO J. 13: 1-7).

Transport of amino acids is another process that is of high importance for the growth of the whole plant and plant organs. As amino acids are indispensable for protein synthesis and other purposes, e.g. as osmolytically active compounds, limitations in their transport via amino acid transporter/permease (AAP) proteins will result in a limitation of plant product yield.

The above mentioned transport and storage processes need highly specific gene expression. If means were available to control expression of genes involved in these processes novel transgenic plants could be produced with desired advantageous properties e.g. enhanced growth, increased yield, resistance to pathogens, larger leaves, and sweeter fruits.

### SUMMARY OF THE INVENTION

The object of the present invention is therefore, to provide means for manipulating expression of plant genes, e.g. to control transport, storage, and growth processes in plants.

This problem is solved by a regulatory element comprising a promoter and a nucleotide sequence, wherein said nucleotide sequence is a nucleotide enhancer and/or repressor sequence comprising a non-coding region of a sucrose transporter gene.

The regulatory element according to the present invention, comprising promoters and nucleotide enhancer and/or repressor sequences, can be used to influence the expression of native or introduced genes. These genes can be of plant or non- plant origin.

In a preferred embodiment of the invention, the nucleotide enhancer and/or repressor sequence is derived from tomato (Lycopersicon esculentum).

Unexpectedly, it was found that a nucleotide sequence from a non-coding region of a SUT gene, especially a nucleotide sequence of intron 1, and/or intron 2, and/or intron 3 of the SUT1 gene, alter gene expression in a way, which is suitable for achieving overexpression or decreased expression of genes in specific cells and tissues.

Thus, a new regulatory element according to the invention drives high expression of genes, stabilizes their mRNA, or decreases gene expression. This can be used to

1. alter expression of sucrose or amino acid transporter genes (more phloem loading at sieve element level, more retrieval along the transport path, more uptake into sink tissues) by expression of SUT or AAP genes in sense orientation.

2. alter the expression of other genes in companion cells, guard cells or trichomes

3. introduce the expression of plant or non-plant genes into companion cells or guard cells or trichomes

4. introduce compounds into the phloem such as RNA and proteins. These can be either new compounds, e.g. from single step pathways to generate e.g. octopine, regulatory RNAs or proteins. This may also be used to trigger systemic acquired suppression to create virus resistant plants or to decrease the expression of target genes

5. introduce or alter the expression of genes in the trichomes to increase plant defense against insects and herbivores or produce molecules with industrial or agricultural value. For example, the expression of monoterpene biosynthetic enzymes in glandular trichomes could be modified. Also, the production of vacuolar flavonoids produced in glandular trichomes that have anti-inflammatory or other pharmacological properties could be manipulated.

6. introduce or alter the expression of genes in guard cells. Natural or synthetic receptors, signal transduction components, ion channels or transporters expressed in guard cells could be used to modify and/or allow control of stomatal aperture regulation. This could increase drought tolerance in dry conditions or increase CO₂ fixation under conditions with sufficient water.

7. introduce xenobiotics into the phloem. Since many compounds applied to plants (xenobiotics such as herbicides or pesticides) are glycosylated, overexpression of a transporter for xenobiotics in companion cells will allow better mobilization of these compounds

8. modify companion cell activities, e.g. enhance ATPase activity and increase raffinose biosynthesis

9. block phloem loading to increase carbohydrate in the leaves e.g. via cosuppression or antisense suppression

Intron 1 of the SUT1 gene decreases gene expression and is considered a repressor of gene expression. Two other introns enhance gene expression and influence the spatial distribution of gene expression. Intron 2, for example, enhances gene expression in guard cells and phloem veins, whereas intron 3 enhances gene expression in trichomes.

According to a preferred embodiment of the present invention, the regulatory element comprises intron 1 of the SUT1 gene.

According to a further preferred embodiment of the present invention, the regulatory element comprises intron 2 and/or intron 3 of the SUT1 gene.

According to another preferred embodiment of the present invention, the regulatory element comprises the sequence of SEQ ID NO: 1, or the complementary sequence of SEQ ID NO: 1, or hybridizes with the nucleotide sequence of SEQ ID NO: 1.

According to another preferred embodiment of the present invention, the regulatory element comprises the sequence of SEQ ID NO: 2, or the complementary sequence of SEQ ID NO: 2, or hybridizes with the nucleotide sequence of SEQ ID NO: 2.

According to another preferred embodiment of the present invention, the regulatory element comprises the sequence of SEQ ID NO: 3, or the complementary sequence of SEQ ID NO: 3, or hybridizes with the nucleotide sequence of SEQ ID NO: 3.

In addition, a 3′-untranslated region (3′UTR) of the SUT1 gene further contributes to the level of expression by stabilizing the mRNA. Therefore, according to a another preferred embodiment of the present invention, the regulatory element further comprises the sequence of SEQ ID NO: 4, or the complementary sequence of SEQ ID NO: 4, or hybridizes with the nucleotide sequence of SEQ ID NO: 4.

Nucleotide sequences of intron 1, intron 2 and intron 3 are given in the sequences with SEQ ID NO: 1, SEQ ID NO: 2, and SEQ ID NO: 3, respectively. A DNA sequence of the 3′UTR is given in the sequence having the SEQ ID NO: 4.

Genes coding for transporter proteins are active in specific regions of the vascular system or in specific tissues/organs, respectively. Gene expression and spatial distribution of gene products depend on promoter activity. Consequently, the use of tissue or organ specific promoters within the novel regulatory element according to the invention allows systematic manipulation of transport, storage and distribution processes for assimilated carbon compounds, e.g. oligosaccharides like sucrose, or amino acids.

Therefore, in a preferred embodiment of the invention, the regulatory element comprises a promoter, which comprises a nucleotide sequence of the SUT1, SUT2, SUT4, AAP3 or AAP4 promoter, or a complementary nucleotide sequence of the SUT1, SUT2, SUT4, AAP3 or AAP4 promoter, or hybridizes with a nucleotide sequence of the SUT1, SUT2, SUT4, AAP3 or AAP4 promoter.

The term hybridization, as it is used in this application, means hybridization under conventional hybridization conditions, as they are described in Sambrook et al. (Molecular Cloning. A laboratory manual, Cold Spring Harbor Laboratory Press, 2^{nd} ed., 1989), preferentially under stringent conditions. According to the present invention, hybridization also means, that, after washing for 20 minutes with 2×SSC and 0,1% SDS at 50° C., preferably at 60° C. and especially preferred at 68° C., or especially for 20 minutes in 0,2×SSC and 0,1% SDS at 50° C., preferably at 60° C. and especially preferred at 68° C., a positive hybridization signal is observed.

Hereinafter, suitable promoters for the purpose of the present invention are described.

SUT1/SUT4 Promoter

The promoter of the high affinity and low capacity sucrose transporter SUT1 is active in companion cells of the phloem. In combination with intron 2, it is also active in guard cells and in combination with intron 3 it is active only in trichomes. Generally it is more active in source tissue (e.g. adult leaves and germinating seed) than in sink tissues (e.g. meristems) or sink organs like roots, tubers, flowers, and fruits. Together with SUT4, SUT1 is responsible for phloem loading. A nucleotide sequence of the LeSUT1 promoter is given in the sequence having the SEQ ID NO: 5.

The promoter of the low affinity and high capacity sucrose transporter SUT4 is specifically active in the minor veins (phloem loading zone) of source leaves. In addition, it is very active in sink tissue. In source leaves, the SUT4 promoter can be used to drive expression of a high affinity sucrose transporter such as SUT1. This could increase sucrose loading into phloem in minor veins concomitant with an increase in sink strength in harvested organs that are sinks (e.g. fruits or seeds). A nucleotide sequence of the Arabidopsis thaliana SUT4 promoter is given in the sequence having the SEQ ID NO: 6.

Overexpression of the gene for the high capacity sucrose transporter SUT4 in source tissue (e.g. leaves) under control of a regulatory element according to the present invention, comprising nucleotide sequences derived from intron 2 and the SUT1 promoter, can increase sucrose loading into the phloem under conditions of high flux rates (e.g. high light intensity). This will lead to increased loading of sucrose into phloem, higher sugar content in the whole sink tissue and especially in roots, fruits, tubers, flowers and seeds, and thus higher total carbohydrate levels in harvested organs. In oil plants, this will lead to higher oil content. Number and dry weight of harvested organs such as fruits, tubers etc. will increase. Overexpression of the SUT4 gene will also be a means to manipulate flowering time and to enhance plant growth in general. What was mentioned above, will also apply to a regulatory element according to the invention with the SUT4 promoter controlling expression of the gene for the high affinity sucrose transporter SUT1.

Cosuppression or antisense suppression of SUT1 and/or SUT4 in source tissue leads to decreased sink strength and results in higher carbohydrate content in source organs. This is important to decrease competition from non-harvested sinks, and to decrease the carbohydrate investment in stems, e.g. to produce more productive dwarf varieties. In addition, this can be used to improve the sweetness of leaf crops such as lettuce and spinach. It will also lead to an accumulation of sugars in source organs and have similar effects as overexpression of SUT4 in non-vascular cells in the leaf. Stem-specific inhibition will reduce stem length and thus increase harvest index.

Overexpression of the SUT4 gene in guard cells under the control of a regulatory element according to the present invention, comprising a nucleotide sequence from intron 2 and the SUT1 promoter, leads to changes in sugar fluxes, and thus energy and osmolyte supply can be used to increase the capacity to open or to reduce the possibility to close stomata. More open stomata will allow better influx of CO₂ for optimal photosynthesis, provided that the water supply is sufficient. It also will allow more frequent opening/closing cycles under conditions of rapidly changing light availability.

Decrease in SUT1 expression in guard cells can be achieved using the SUT1 promoter and intron 2 to express the SUT1 gene in the antisense orientation. This would block stomatal opening, e.g. during the day, and increase water use efficiency since less water is lost and thus improve plants normally not growing in dry conditions with respect to drought tolerance.

Cosuppression or antisense suppression of sucrose transporter genes will prevent efficient phloem loading and will increase sugar content in leaves. Thus the result will be bigger leaves, with more defense potential against pathogens, especially since defense genes are upregulated by increased sugar content, a thicker cuticle and more secondary metabolites, e.g. higher precursor content for production of biodegradable plastics (e.g. PHB). Both the SUT1 and SUT4 promoters alone or in combination with other cis elements, e.g. intron 2, are suitable for this purpose.

Genes under the control of a regulatory element according to the invention, comprising a nucleotide sequence derived from intron 3 and the SUT1 promoter, will be expressed exclusively in trichomes. This could be used to produce plants with modified trichomes, e.g. trichomes containing special plant products such as defense compounds (e.g. pesticides) or other compounds. This could also be used to express genes encoding transporters or other proteins in trichomes that would enhance accumulation of metals and improve the utility of transgenic plants in phytoremediation.

Overexpression of genes such as SUT1 in seeds will result in an improved germination rate. A suitable promoter is the SUT4 promoter.

Expression of both the SUT4 gene under the control of a regulatory element, comprising the SUT1 promoter and nucleotide sequences derived from intron 2, and the SUT1 gene under control of a regulatory element according to the invention with the SUT4 promoter may result in plants with very high yields.

Since higher sugar transport in the phloem is often negatively correlated with amino acid transport, overexpression of the SUT1 and/or SUT4 gene may lead to reduced amino acid content in sink tissues, e.g. in potato tubers or sugar beet tap roots. However, overexpression of amino acid transporter genes under control of a regulatory element according to the invention will result in fruits with higher amino acid and/or protein content (e.g. in soy beans and other legumes).

SUT2 Promoter

In young plants, the SUT2 promoter is active in the whole plant. In older plants, promoter activity is found in roots, in major veins of plant leaves, in sepals and anthers. A nucleotide sequence of the Arabidopsis thaliana SUT2 promoter is given in the sequence having the SEQ ID NO: 7.

The SUT2 promoter can be used to express plant or non-plant genes in phloem and other tissues. This could be used to increase yield or nutrient content of harvested tissues.

The SUT2 gene encodes a sucrose sensor. The SUT2 promoter can be used to decrease the expression of the SUT2 gene by expression of SUT2 in the antisense orientation or by overexpression of SUT2 leading to cosuppression. Decreased expression of SUT2 would modify sucrose sensing and sucrose transport due to SUT2 and/or other sucrose transporters (e.g. SUT1 and SUT4). Modification of sucrose transport activity could be used to increase carbon partitioning to harvested organs as suggested above.

AAP3 Promoter

The promoter of the amino acid transporter AAP3 is mainly active in the stele of roots, in flowers and in cotyledons. A nucleotide sequence of the Arabidopsis thaliana AAP3 promoter is given in the sequence having the SEQ ID NO: 8.

The AAP3 gene encodes an amino acid transporter. The AAP3 promoter can be used to decrease the expression of the AAP3 gene by expression of AAP3 in the antisense orientation or by over expression of AAP3 leading to cosuppression. Decreased expression of AAP3 would modify translocation of amino acids between the xylem and phloem in roots and would decrease amino acid translocation to the shoot. This can be used to decrease the concentration of toxic or undesirable nitrogenous compounds in the shoot.

AAP4 Promoter

The promoter of the amino acid transporter AAP4 is active in pollen and tapetum tissue. It is also active in major veins of the phloem of mature leaves, stem and roots. A nucleotide sequence of the Arabidopsis thaliana AAP4 promoter is given in the sequence having the SEQ ID NO: 9.

The AAP4 gene encodes an amino acid transporter. The AAP4 promoter can be used to express plant or non-plant genes in pollen or tapetal tissue. This could be used to generate male sterile plants or to limit cross pollination from transgenic plants (to limit transgene dissemination). Exemplary, the AAP4 promoter can be used to decrease the expression of the AAP3 gene by expression of AAP3 in the antisense orientation or by over expression of AAP3 leading to cosuppression. This could inhibit pollen function and lead to male sterile plants. This would be important for plant breeding.

A regulatory element according to the invention can be used to control the expression of a variety of plant and non-plant genes, comprising, but not restricted to, the SUT1, SUT2, SUT4, AAP3 and AAP4 gene. Thus, it can be used to influence complex transport and distribution processes, e.g. for nutritive substances like oligosaccharides and amino acids, vitamins, minerals, or growth regulatory substances such as peptide hormones or other hormones within the plant.

The invention also relates to a promoter for use in a regulatory element according to the invention, wherein the promoter is one of the SUT1, SUT2, SUT4, AAP3 and AAP4 promoters.

The invention also relates to vectors or mobile genetic elements, comprising a regulatory element according to the invention. Suitable vectors or mobile genetic elements, e.g. viruses, bacteriophages, cosmids, plasmids, yeast artificial chromosomes, T-DNA, transposable elements, insertion sequences etc., to introduce nucleotide sequences into host cells are well known to one skilled in the art of molecular cloning techniques.

Further, the invention relates to host cells like bacterial cells, yeast cells or plant cells. In a preferred embodiment of the invention, plant cells, especially of the genus Beta vulgaris, are transformed with a regulatory element according to the invention.

The invention also relates to plants, parts thereof, and seeds of plants, which comprise, or are derived from, cells transformed with a regulatory element according to the invention. Plants, transformed with a regulatory element according to the invention, can be elected from the group of plants comprising, but not restricted to, Pinidae, Magnoliidae, Ranunculidae, Caryophyllidae, Rosidae, Asteridae, Aridae, Liliidae, Arecidae, and Commelinidae (subclasses according to Sitte, P., Ziegler, H., Ehrendorfer, F., Bresinsky, A., eds., 1998, Strasburger—Lehrbuch der Botanik fur Hochschulen; 34. ed., Gustav Fischer Verlag, Stuttgart). Thus, a transgenic plant according to the invention may be elected from the group of plants comprising, but not restricted to, sugar beet (Beta vulgaris), sugar cane, Jerusalem artichoke, Arabidopsis, sunflower, tomato, tobacco, corn, barley, oat, rye, rice, potato, rape, cassava, lettuce, spinach, grape, apple, coffee, tea, banana, coconut, palm, pea, bean, pine, poplar, and eucalyptus.

Further, the invention relates to a process for the production of a transgenic plant, especially of the genus Beta vulgaris, comprising the steps of transforming a plant cell with a regulatory element according to the invention and regenerating a plant therefrom. These steps can be performed using standard procedures.

The sequence protocol includes:

SEQ ID NO: 1: A nucleotide sequence of intron 1 of the SUT1 gene from Lycopersicon esculentum

SEQ ID NO: 2: A nucleotide sequence of intron 2 of the SUT1 gene from Lycopersicon esculentum

SEQ ID NO: 3: A nucleotide sequence of intron 3 of the SUT1 gene from Lycopersicon esculentum

SEQ ID NO: 4: A nucleotide sequence of a 3′untranslated region of the SUT1 gene from Lycopersicon esculentum

SEQ ID NO: 5: A 2,3 kb nucleotide sequence of the SUT1 promoter from Lycopersicon esculentum

SEQ ID NO: 6: A 3,1 kb nucleotide sequence of the SUT4 promoter from Arabidopsis thaliana

SEQ ID NO: 7: A 2,4 kb nucleotide sequence of the SUT2 promoter from Arabidopsis thaliana

SEQ ID NO: 8 A 2,5 kb nucleotide sequence of the AAP3 promoter from Arabidopsis thaliana

SEQ ID NO: 9 A 2,7 kb nucleotide sequence of the AAP4 promoter from Arabidopsis thaliana

### BRIEF DESCRIPTION OF DRAWINGS

The following figures serve to elucidate the invention. It shows:

FIG. 1 A schematic drawing of promoter deletion (promoter-GUS) constructs for LeSUT1. In this:

HindIII, SalI, EcoRI, BamHI, XbaI, EcoRV, SmaI: Cleavage positions of restriction enzymes.

ATG: Translation start

uidA: GUS gene

npt II: Neomycin-Phosphotransferase II gene

nT: nopaline sythase terminator

nP: nopaline sythase promoter

LB: left border sequences (from T-DNA)

RB: right border sequences (from T-DNA)

kb: kilobases

p: promoter

FIG. 2 A schematic drawing of promoter-SUT1-GUS constructs for LeSUT1. In this:

HindIII, SalI, EcoRI, BamHI, XbaI, EcoRV, SmaI, BclI,

SstI: Cleavage positions of restriction enzymes.

ATG: Translation start

uida: GUS gene

npt II: Neomycin-Phosphotransferase II gene

nT: nopaline sythase terminator

nP: nopaline sythase promoter

LB: left border sequences (from T-DNA)

RB: right border sequences (from T-DNA)

E1, E2, E3, E4: Exons 1 to 4

I1, I2, I3: Introns 1 to 3

3′UTR: 3′untranslated region

FIG. 3 A. Comparison of average β-glucuronidase activities of promoter-GUS, promoter-SUT1-GUS and promoter-SUT1-GUS-3′UTR constructs.

B. Percent of plants, transformed with promoter-GUS, promoter-SUT1-GUS and promoter-SUT1-GUS-3′UTR constructs, showing β-glucuronidase activity in different activity intervals.

FIG. 4 Schematic drawing of promoter-intron-GUS-3′UTR constructs for LeSUT1 In this:

HindIII, SalI, EcoRI, BamHI, XbaI, EcoRV, XhoI SmaI:

Cleavage positions of restriction enzymes.

ATG: Translation start

uida: GUS gene

npt II: Neomycin-Phosphotransferase II gene

nT: nopaline sythase terminator

nP: nopaline sythase promoter

LB: left border sequences (from T-DNA)

RB: right border sequences (from T-DNA)

I1, I2, I3: Introns 1 to 3 3′UTR: 3′untranslated region

### DETAILED DESCRIPTION OF THE INVENTION

The invention is further defined, by way of illustration only, by reference to the following examples.

### EXAMPLE 1

### Expression Analysis of the Sucrose Transporter LeSUT1 by the GUS Fusion System

The spatial expression within the plants was uncovered by a promoter-reporter-gene-fusion using the GUS (β-glucuronidase) gene fusion system (Jefferson et al. 1987, EMBO J. 6: 3901-3907). First, the promoter of the SUT1 gene from tomato was isolated.

For the construction of promoter-GUS constructs, the genomic clone of LeSUT1 was isolated by screening a genomic library, containing genomic DNA from Lycopersicon esculentum cv. VFN8 in the EMBL-3 vector (Clontech). By hybridization of a ³²p-labeled StSUT1 probe (from potato, Solanum tuberosum) under low stringency conditions 11 positive λ-phages could be identified. The 7 strongest hybridizing phage isolates were used to obtain λ-phage DNA by plate lysates for a restriction analysis. The 2 phages that gave the strongest signal (10/1 and 2/2) were analyzed in more detail by Southern blot analysis. A 2.1 kb BamHI fragment was isolated that, compared with the restriction pattern of the genomic clone, contains the promoter and the 5′-region of LeSUT1. This BamHI fragment was cloned into pON 184 and sequencing showed the correspondence with the 5′-end of the LeSUT1 cDNA. The translation start of LeSUT1 is at position 1693 in this fragment. The transcription start determined by comparison with the cDNA start of LeSUT1 is at position −60 of the first ATG of LeSUT1 (at position 1633 of the fragment).

For the generation of a 1.7 kb promoter-GUS construct the 2.1 kb BamHI clone was used. Because of the lack of a suitable restriction site in the N-terminal region of LeSUT1 a 840 bp fragment of the promoter was amplified by polymerase chain reaction (PCR) using the reversed primer 5′-GGGGTACCCGGGTGTACCATTCTCCATTTT-3′ containing restriction sites for SmaI 15 bp downstream of the first ATG of LeSUT1 and for KpnI behind the SmaI site. The forward primer 5′-CCGATATCTCAATTGGTT-3′contained a EcoRV site which is located at position 887 in the 2.1 kb fragment. The PCR product was cloned EcoRV/KpnI into pON184 containing the 850 bp BamHI/EcoRV fragment of the 5′-end of the LeSUT1 promoter region. Afterwards the 1.7 kb promoter fragment was cloned BamHI/SmaI into the plant binary vector pBI101.3 (Jefferson et al. 1987, EMBO J. 6: 3901-3907). The reading frame of this translational fusion was checked by sequencing the uida (GUS) gene. In this construct the first five amino acids are encoded by LeSUTl, the following seven amino acids are encoded by the polylinker of pBI101.3, followed by the uida gene.

Expression of GUS under control of the 1.7 kb promoter fragment of LeSUT1 showed the same expression pattern, restricted to the vascular tissue in source leaves of transgenic potato and tomato plants. Unexpected in this case was the low rate of plants expressing GUS. For both species potato and tomato, the rate of transgenic plants showing GUS expression was only about 20%. This indicates a positional effect in which the promoter-GUS construct was incorporated near an enhancer. Therefore, the 1.7 kb promoter itself has low activity. In addition, the intensity of GUS derived blue staining in the veins was unexpectedly low except for one or two transgenic lines showing strong expression. Compared to Northern blot analysis showing very strong expression of SUT1 in source leaves (Riesmeier et al. 1993, Plant Cell 5: 1591-1598), the expression of GUS under the control of the SUT1 promoter was weak.

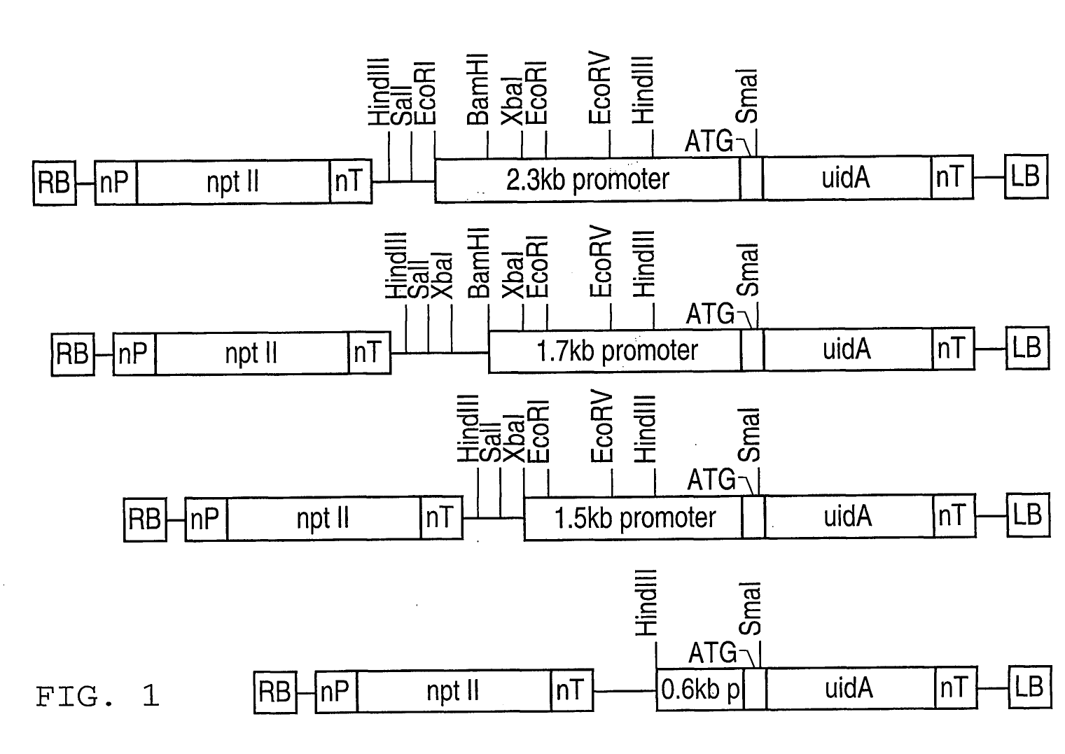

Therefore, it was tested whether an inhibiting element was present in the 1.7 kb promoter fragment or whether in the 1.7 kb fragment important upstream cis elements, responsible for the expression of SUT1, were missing. On the one hand, further upstream promoter sequences were isolated to generate a larger 2.3 kb promoter construct and on the other hand the 1.7 kb fragment was shortened to create an 1.5 kb and an 0.6 kb promoter construct (FIG. 1).

To isolate an extended promoter region of LeSUT1 a DIG-labeled probe (Boehringer) of the 5′-end of the 2.1 kb BamHI fragment was hybridized with DNA of the phage lysates 10/1 and 2/2 cut with different restriction enzymes. A positive 0.8 kb EcoRI fragment from phage 10/1 was cloned into pBlueskript SK⁺ (Stratagene). Sequencing of this fragment showed the correspondence with the 5′-end of the 2.1 kb BamHI fragment and no homology to the EMBL-3 vector.

1.5 kb promoter-GUS construct:

For the generation of the 1.5 kb promoter-GUS construct a 0.2 kb XbaI fragment from the 5′-end of the LeSUT1 promoter was deleted from the 1.7 kb promoter-GUS construct and the remaining construct was religated.

0.6 kb promoter-GUS construct:

For the generation of the 0.6 kb promoter-GUS construct a 1.1 kb HindIII fragment from the 5′-end of the LeSUT1 promoter was deleted from the 1.7 kb promoter-GUS construct and the remaining construct was religated.

2.3 kb promoter-GUS construct:

For the generation of the 2.3 kb promoter-GUS construct the 0.8 kb EcoRI fragment of the extended 5′-region of the LeSUT1 promoter was cloned into the EcoRI site of pBlueskript SK⁺. This fragment was then cut with SalI/XbaI and cloned into the SalI/XbaI digested 1.7 kb promoter-GUS construct.

The expression of these promoter-GUS constructs was investigated in Nicotiana tabacum. The specificity of promoters in different plant species are maintained.

The translational promoter-GUS fusions were introduced into the genome of Nicotiana tabacum via Agrobacterium mediated gene transfer. After selective regeneration of plants on kanamycin containing media leaf discs of plants from sterile culture were tested for β-glucuronidase (GUS) activity.

In Table 1, the rates of plants showing β-glucuronidase activity is presented. For the 2.3 kb, 1,7 kb and 0.6 kb promoter constructs the rates are similar between 40-50%.

TABLE 1
β-glucuronidase (GUS) activity in transgenic LeSUT1
promoter-GUS tobacco lines (after 2 days incubation in X-Gluc
solution).

| | No. of | No. of plants | % of plants |
|---|---|---|---|
| | transgenic | showing GUS- | showing GUS- |
| Construct | plants | staining | staining |
| 2.3P-GUS | 41 | 16 | 39 |
| 1.7P-GUS | 36 | 14 | 39 |
| 1.5P-GUS | 31 | 5 | 16 |
| 0.6P-GUS | 36 | 19 | 50 |

Expression was lowest in plants containing the 1.5 kb promoter construct. The intensity of average staining was very low for all constructs after 2 days of incubation in the X-Gluc solution. The pattern of the weak blue staining was in most cases restricted to the major veins for all four constructs.

### Influence of Intragenic Sequences (Introns) and the 3′UTR of LeSUT1 on the Expression of GUS

None of the deletion constructs resulted in expected high expression rates and levels of the β-glucuronidase. The absence of required further upstream promoter sequences is unlikely. Most plant genes require shorter promoters for their expression. More likely is that in the case of LeSUT1 other sequences downstream of the translation start are required for sufficient gene expression.

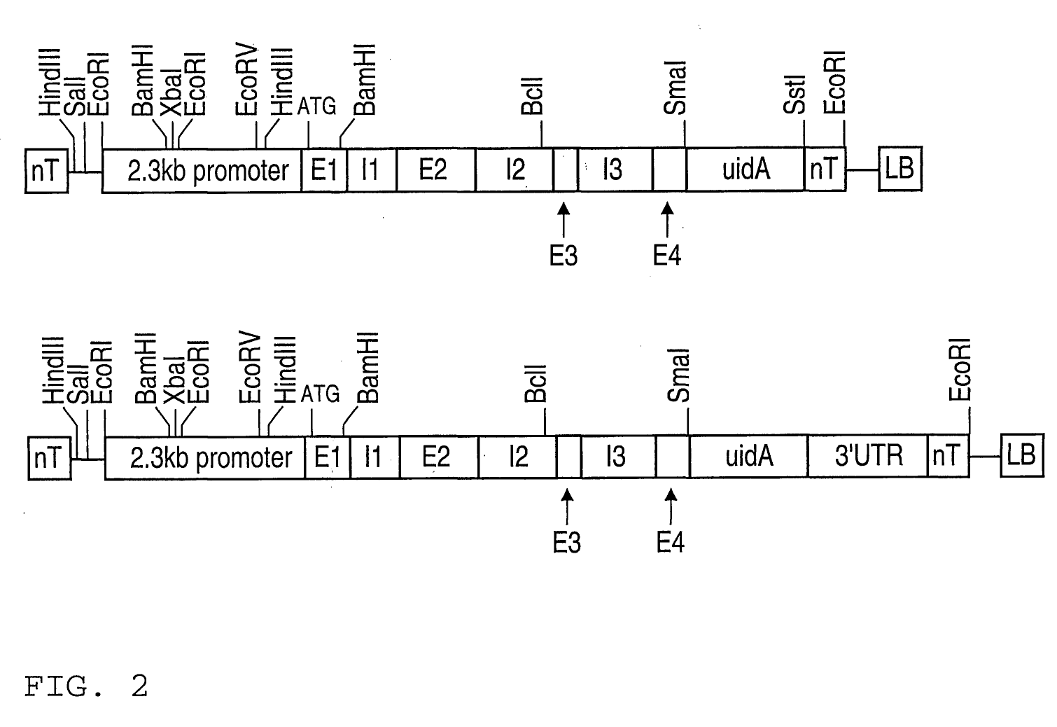

To figure out if intragenic sequences play a role in SUT1 expression two further constructs were generated (FIG. 2). In the one construct the longest promoter fragment (2.3 kb) together with the entire coding region (including introns) of LeSUT1 ending 6 amino acids before the stop codon was fused to the GUS gene. LeSUT1 has four exons and three introns. The second construct contains in addition an approx. 1.2 kb fragment containing the 3′UTR of LeSUT1 placed between the GUS gene and the nopaline synthase terminator (FIG. 2).

2.3 kb promoter-SUT1-GUS construct:

For the generation of a construct that, in additionally to the promoter region, contains the whole genomic sequence of LeSUT1, a genomic 7.1 kb EcoRI fragment containing 1.5 kb of the promoter region, the whole genomic sequence (4 exons and 3 introns) of the SUT1 gene and further upstream sequence was cloned into pBlueskript SK⁺ with the promoter sequence orientated to the SalI site of the polylinker of the vector. Because of a lacking suitable restriction site around the stop codon of SUT1 a 1.4 kb fragment was amplified by PCR. The reversed primer LeSUT1-1394rev (5′-GAAACCGCCCATCCCGGGTGGTGGTTTAG-3′) contained a SmaI site 18 bp in front of the SUT1 stop codon, the forward primer LeSUT1-1394for (5′-GTGGGCTTGTAAACGGTTGTAAGTCAC-3′) was designed in the middle of the second intron 357 bp in front of a BclI site at position 2764 behind the first ATG of SUT1. This PCR product was then cut with BclI and SmaI and cloned into the 7.1 kb EcoRI fragment in pBlueskript SK⁺ that had been digested with BclI and SmaI. Afterwards, to this clone, the 0.8 kb EcoRI fragment containing the 5′-end of the LeSUT1 promoter region was cloned into the EcoRI site. The final 6.0 kb fragment containing 2.3 kb of the promoter region and the genomic sequence ending 6 amino acids before the stop codon of LeSUT1 was then cloned into the SalI and SmaI digested plant binary vector pBI101.3 (Jefferson et al. 1987, EMBO J. 6: 3901-3907). The maintenance of the reading frame was checked by sequencing into the SmaI fusion site by using the primer 5′uidA.

2.3 kb promoter-SUT1-GUS-3′UTR construct:

The 3′UTR of LeSUT1 was isolated by PCR using the forward primer 5′-TTCCGGCCGAAAAAATTACAAAAGACGAGGAAG-3′ containing a EagI site and the reversed primer 5′-TACCGAGCTCCTAGGCGAGGTCGACGGTAT-3′ containing a SalI site on the 7.1 kb genomic EcoRI clone of LeSUT1. The 1.2 kb product was cut with EagI/SalI and cloned into pBlueskript SK⁺. To minimize possible PCR errors approximately 1 kb of the C-terminal sequence of the PCR-product in pBlueskript SK⁺ was replaced by genomic sequence from the 7.1 kb EcoRI clone by using BstI that cuts at position 212 of the 3′UTR and SalI that cuts upstream of the 7.1 kb EcoRI fragment in the pBlueskript SK+vector. The fragment was then cut out with EagI and EcoRV. The 5′ overhang of the EagI site was filled in with the Klenow enzyme. This fragment with blunt ends was then cloned into the SstI site between the uida gene and the nos terminator of pBI101.3. The 6.0 kb fragment, containing 2.3 kb of the promoter region and the genomic sequence ending 6 amino acids before the stop codon of LeSUT1 was then cut with SalI and SmaI and cloned into pBI101.3 containing the 3′UTR.

### Quantitative Effects of Intragenic Sequence and the 3′UTR of LeSUT1 on β-glucuronidase Activity in Nicotiana tabacum

The GUS fusions were introduced into the genome of Nicotiana tabacum and β-glucuronidase activity was first analyzed in transgenic tobacco plants from sterile culture. For the 2.3P-SUT1-GUS construct 20 transgenic plants were obtained of which 11 plants showed GUS expression. These 55% of transgenic plants showed a clearly stronger and more specific blue staining on average than the plants transformed with any of the promoter deletion constructs. For the 2.3P-SUT1-GUS-3′UTR construct 46 transgenic plants were obtained of which 36 (78%) plants showed GUS expression. The expression level determined as intensity of blue staining in these plants appeared to be the highest on average.

To quantify the visual impression of higher GUS expression for these constructs the activity of the β-glucuronidase enzyme in the transgenic plants was determined (Jefferson et al. 1987, EMBO J. 6: 3901-3907). For this, all transgenic tobacco lines of the 2.3P-GUS, the 2.3P-SUT1-GUS and the 2.3P-SUT1-GUS-3′UTR constructs were transferred into the greenhouse to grow them under more physiological conditions compared to those in sterile culture. After 10-12 weeks after transfer into the greenhouse fully expanded big source leaves were cut of the plant approx. 1 cm below the leaf base in the petiole and frozen in liquid nitrogen. To achieve an equal distribution of vascular tissue and leaf mesophyll tissue the leaves were first ground in liquid nitrogen to a fine powder. Aliquots of this powder were then used to determine the P-glucuronidase activity in the leaves using the standard fluorimetric GUS assay (Gallagher, S. R., 1992, in GUS protocols: Using the GUS gene as a reporter of Gene expression, Gallagher, S. R., (ed.), Academic Press, Inc., pp 47-59; Jefferson et al. 1987, EMBO J. 6: 3901-3907).

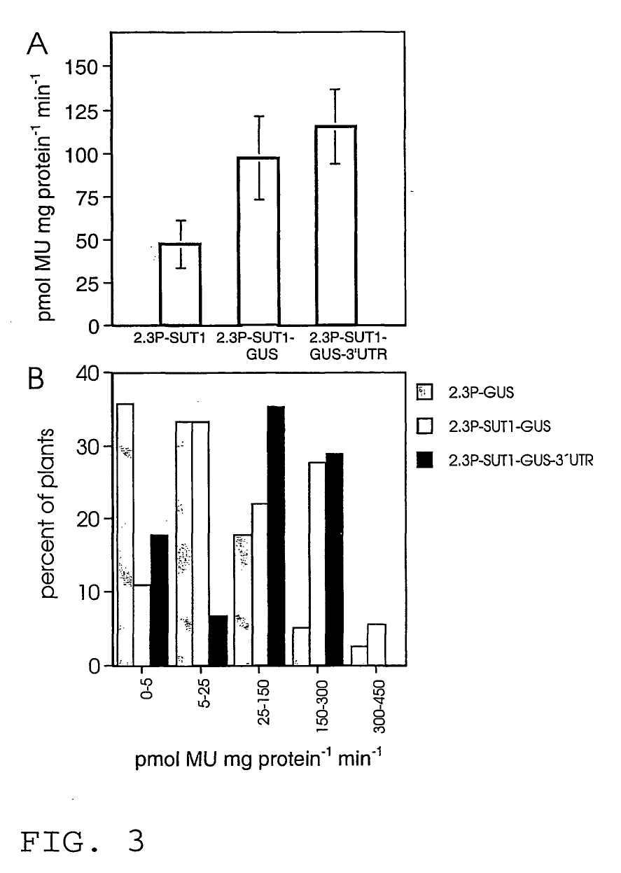

The activity was determined as the average activity of the β-glucuronidase (FIG. 3A) for all plants and the values were separated in percent of plants showing enzyme activity in different activity intervals (FIG. 3B).

The activity of the β-glucuronidase was nearly 50 pmol MU mg protein⁻¹ min⁻¹ for the 2.3P-GUS construct (FIG. 3A). The activity of GUS in plants containing the 2.3P-SUT1-GUS construct was about two-fold higher in average and the highest activity in average could be observed for the 2.3P-SUT1-GUS-3′UTR construct with an activity of about 120 pmol MU mg protein⁻¹ min⁻¹. The higher activity of this construct compared with the 2.3P-SUT1-GUS construct is not significant (t-test, α=0.05). Nevertheless, visually the GUS expression appeared to be stronger on average.

The distribution of β-glucuronidase activities into different intervals shows that the vast majority (70% of all plants) of the 2.3P-GUS plants have enzyme activities in a range of 0-25 pmol MU mg protein⁻¹ min⁻¹. The distribution of the 2.3P-SUT1-GUS plants is clearly shifted to a majority of plants showing activity between 5 and 150 pmol MU mg protein⁻¹min⁻¹ (56% of all plants). The clear majority of the 2.3P-SUT1-GUS-3′UTR plants show an activity of 25-300 pmol MU mg protein⁻¹ min⁻¹ (65% of all plants).

This distribution of the different enzyme activities for the different constructs indicate both an enhancing effect of the exons and/or introns and the 3′UTR on the activity of the LeSUT1 promoter.

### Histochemical Analysis of Heterologous Expression of the 2.3P-SUT1-GUS-3′UTR Constructs in Nicotiana tabacum

The expression pattern in the 2.3P-SUT1-GUS and the 2.3P-SUT1-GUS-3′UTR tobacco lines were similar. Of 36 2.3P-SUT1-GUS-3′UTR lines 34 showed the same expression pattern with varying intensities. 2 plants showed a different staining pattern. The 11 2.3P-SUT1-GUS lines showed the same average expression pattern as the 3′UTR construct with lower intensity in general.

The expression pattern in leaves of tobacco plants grown in sterile culture was clearly different compared with plants expressing GUS under the control of the 2.3 kb promoter fragment only. GUS expression was observed in all leaf veins, in trichomes and in guard cells. The staining in the minor veins was not equally distributed within the vein but appeared to be concentrated in spots. The expression in leaves of plants grown in the greenhouse was the same.

The GUS-expression in the vascular tissue was further investigated in thin sections to determine in which cell type the GUS expression is localized. GUS staining was detectable in the internal phloem and stronger in the external phloem of mid veins. In longitudinal midvein sections the staining appeared to be concentrated in spots along the phloem. In cross sections of minor veins GUS expression was clearly detectable in companion cells. In sieve elements of minor veins no GUS expression was observed. In longitudinal sections of midveins GUS expression was strongest in companion cells, concentrated around the nuclei. In some cases little staining was also detectable in sieve elements.

### Histochemical Analysis of Heterologous Expression of LeSUT1 2.3P-intron-GUS-3′UTR Constructs in Nicotiana tabacum

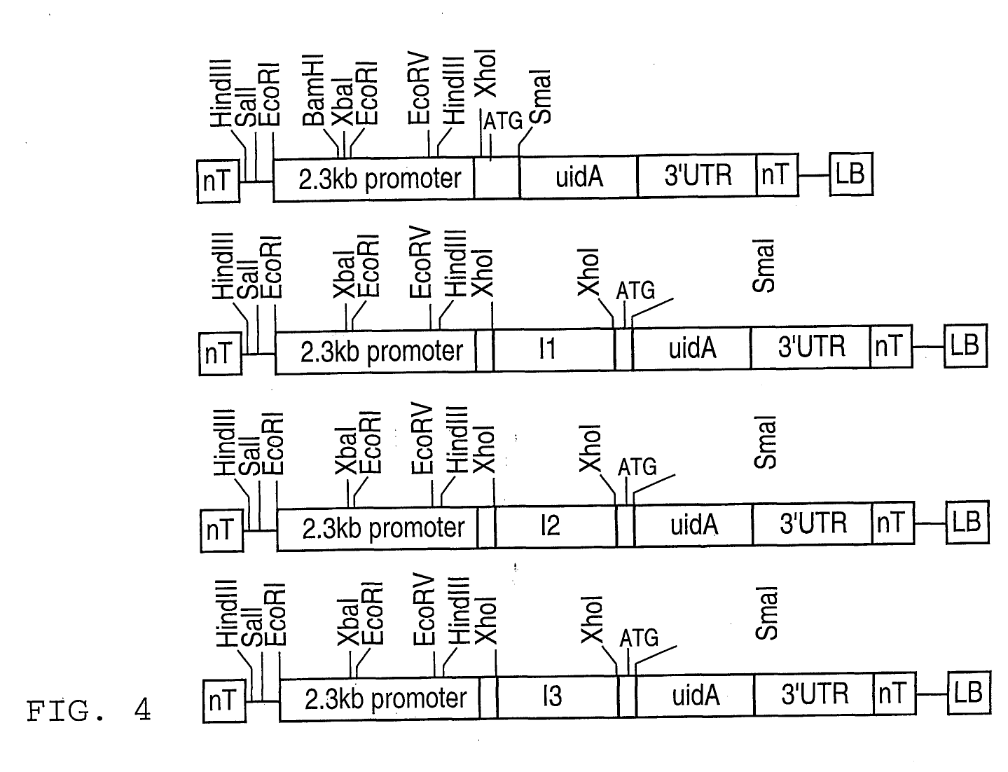

To reveal the function of the introns on LeSUT1 expression, further constructs were made (FIG. 4). To allow a high efficiency of intron splicing the 5′UTR was chosen for the intron insertion. To maintain the enhancing effect of the 3′UTR, the 3′UTR was kept in the intron constructs between the GUS gene and the nopaline synthase terminator. The introduction of the necessary XhoI restriction site and the maintenance of the intron border sequences resulted in little sequence modifications within the 5′UTR of LeSUT1.

To insert introns into the 5′UTR of LeSUT1 a restriction site was introduced for XhoI which does not cut in any of the introns, the plant binary vector, the 2.3 kb promoter fragment, or the 3′UTR of LeSUT1. Using PCR, a 650 bp fragment of the 3′ end of the 2.3 kb promoter was amplified using the reversed primer LeSUT1PXhoIrev (5′-TTTCCCGGGTGTACCATTCTCCATTTTTTTTTCTTCTAAGAAACTAAAATTGCTCGAGTT TAATTTTGGG-3′) containing the previously introduced SmaI site and, further upstream, a XhoI site leading to an exchange of three basepairs and the forward primer LeSUT1PXhoIfor (5′-GATAAATCAAGGTGATATATGTACATAC-3′) containing an endogenous Bsp4107I site which cuts twice in the uida gene. Therefore, the PCR product was then used in a triple ligation step together with the excised 5′SalI/Bsp1407I fragment of the 2.3 kb promoter fragment and the SalI/SmaI cut pBI101.3 vector containing the 3′UTR of LeSUT1.

The three introns were amplified using PCR. The following primers, containing XhoI sites at both ends, were used:

For Intron 1:
LeSUT1intron1for
5′-GCTAATAACATGCTCGAGGTAATTTTCAAATCG-3′
LeSUT1intron1rev
5′-CCAGTAGTGCTCTGCTCGAGCCCTG-3′
For Intron 2:
LeSUT1intron2for
5′-CCCTTGGTATTCCTCGAGCGGTGAGTTTC-3′
LeSUT1intron2rev
5′-CCAAAGCAAATGGAATACCTCGAGTTACCTG-3′
For Intron 3:
LeSUT1intron3for
5′-CTTGCAATTGTTGTACTCGAGGTAC-3′
LeSUT1intron3rev
5′-CTTACTAGTGACCTCGAGATCTATATC-3′

The introns were then cloned into the XhoI digested 5′UTR of the 2.3PXhoI-GUS-3′UTR construct. The correct orientation of the introns was proven by sequencing.

In addition to the intron constructs the construct with the XhoI modified 5′UTR was also introduced into the genome of tobacco plants as a control. For the intron 1 construct 30 kanamycin resistant plants were obtained, whereas 8 were obtained for the intron 2 construct, 20 were obtained for the intron 3 construct, and 24 were obtained for the control construct. Leaves from sterile culture grown plants were incubated over night in X-Gluc solution at 37° C.

17 plants containing the intron 3 construct showed GUS activity (=85%) of which in 16 plants blue staining could be exclusively detected in the trichomes. One plant showed an overall GUS expression in the leaf. Six plants containing the intron 2 construct showed GUS expression (=75%). The expression in 6 of these plants was restricted to the guard cells and veins. Two plants showed no staining.

In seven tobacco lines (=30%) transformed with the control construct weak, non-specific GUS staining was observable. In general, the staining pattern was similar to those plants that had been transformed with the 2.3 P-GUS construct.

The results indicate a clear enhancing and/or spatial regulation function for intron 3 and intron 2. The presence of intron 3 is necessary to drive high level gene expression in trichomes whereas intron 2 represents the element required for gene expression in veins and guard cells.

Intron 1 caused a negative effect on gene expression. Of 24 plants transformed with the control construct, only 7 (30%) showed GUS staining. However, none of the plants (of 30) containing the Intron 1 construct showed visible GUS staining. This indicates, that intron 1 has a negative regulatory effect on gene expression. Therefore, the introduction of intron 1 into other genes, particularly into existing introns may down-regulate expression. This represents a novel strategy to regulate expression.

The comparable expression of the control construct to the 2.3P-GUS constructs indicates that at least the modification of the 5′UTR by introducing the XhoI site did not affect the expression level. Furthermore, this result indicates that the 3′UTR is not sufficient, in combination with the promoter, to provide strong expression observed for the 2.3P-SUT1-GUS-3′UTR construct.

### EXAMPLE 2

### Expression Analysis of the Sucrose Transporter Gene SUT2 by Promoter-GUS Fusion in Arabidopsis

The promoter of AtSUT2 was isolated by polymerase chain reaction (PCR) using Pfu-polymerase and the primers AtSUT2Pfor (5′-ACGCTTGTCGACCCGGCTCTATCACGTTAACAC-3′ and 5′-ACGCTTGTCGACCGTTTGAGAAATGACGAAGGAG-3′ for the 2.2 kb and 1.2 kb promoter fragments, respectively) and AtSUT2Prev (5′-GTCCCCCGGGCAACACACAGATCCCTAATTCG-3′) on genomic DNA of Arabidopsis thaliana Col-O ecotype. Transcriptional fusions were generated by cloning 2.2 kb and 1.2 kb promoter fragments into the SalI/SmaI site of PGPTV-HPT (Becker et al. 1992, Plant Mol. Biol. 20: 1195-1197). Arabidopsis was transformed by vacuum infiltration using transformed Agrobacterium GV2260 (Clough and Bent 1998, Plant J. 16: 735-743). Among 10 hygromycin resistant transformant lines for the 1.2 kb promoter construct 9 showed the same expression pattern. For the 2.2 kb promoter construct 2 plants were regenerated both showing the same expression as the 1.2 kb promoter constructs.

In young seedlings and plants GUS expression was found in all tissues of the shoot and roots. In older greenhouse grown plants GUS expression in source leaves was restricted to the major veins and hydathodes. In stem, no GUS expression was detectable. In flowers, GUS expression was detected in sepals, anthers, the stigma of the pistil and at the peduncel.

### EXAMPLE 3

### Expression Analysis of AtSUT4 in Arabidopsis by Promoter-GUS Fusions

To analyze the expression of AtSUT4 in plants, the promoter was isolated and fused to the GUS reporter gene (Jefferson et al. 1987, EMBO J. 6: 3901-3907). A 3.1 kb, a 2.2 kb and a 1.1 kb fragment was isolated by polymerase chain reaction on genomic DNA of Arabidopsis thaliana Col-O using Pfu polymerase and transcriptionally fused to the GUS gene in the plant binary vector PGPTV-HPT (Becker et al. 1992, Plant Mol. Biol. 20: 1195-1197). For the isolation of the promoter fragments the following oligonucleotides were used as primers:

AtSUT4Prev
5′-TCCCCCGGGCTCGCTTCACAGTCGTCGTGGCGTAG-3′
AtSUT4P3.1kbfor
5′-GTTTGTTGTCGACGGGCGAAATCTCGCATAACTTC-3′
AtSUT4P2.2kbfor
5′-ACGGTCGACAGGGTCGCATCTCGATATTATGG-3′
AtSUT4P1.1kbfor
5′-ACGCTTGTCGACGACCCGGTGAGTAATTGAACGC-3′

For the 3.1P-GUS construct, 17 plants could be regenerated on hygromycin containing media of which 14 showed an identical GUS expression, for the 2.2P-GUS construct 12 plants were obtained of which 8 showed an identical expression of GUS. The expression pattern for the 3.1P-GUS and the 2.2P-GUS constructs were the same. No transformants were obtained for the 1.1P-GUS construct.

In germinating seed and in very young seedlings an overall expression of GUS was found. In the developing young plant the staining was more specific, with highest activity in the center of the rosette and sink leaves, and after transition to source leaves expression was restricted to the minor veins. This expression pattern is in contrast to LeSUT1 from tomato, which drives expression in all veins of source leaves.

In the inflorescence, GUS activity was found within developing flowers in the anther and pistil. At anthesis, staining was restricted to the anthers. In mature flowers GUS activity was hardly detectable. After pollination GUS activity was found throughout the developing silique and was relatively strong in the funiculi and the ingrown pollen tubes.

In general, GUS activity in flowers and fruits was restricted to the growing organs still in development without high specificity, indicating a role for AtSUT4 in growing sink organs for direct sucrose uptake into sink cells.

GUS expression was never found in stems except in the axils of branches. GUS activity remained strongest throughout development in the center of the plant rosette.

GUS expression in roots was strongest at the point of lateral root initiation. GUS activity was also detectable in the vascular system with stronger activity at the branches of lateral roots.

### EXAMPLE 4

### Expression Analysis of AtAAP3 in Arabidopsis by Promoter-GUS Fusions

To investigate the expression of AAP3 pattern, a 7 kb AAP3 promoter was fused to the GUS gene and introduced into tobacco (Fischer et al. 1995, J. Biol. Chem. 270: 16315-16320, Fischer 1997, Dissertation, Eberhard-Karls University, Tuebingen). However, the expression of GUS activity was not stable, most of the transformed lines showed no staining at all. This might be due to the length of the promoter used. Therefore, two promoter-GUS fusion constructs which carry 1.5 kb and 3.5 kb of the AAP3 promoter were constructed. Arabidopsis and Nicotiana plants were transformed with these constructs. Transgenic lines were obtained and analyzed from 1.5 kb (1.5P-GUS) and 3.5 kb (3.5P-GUS) promoter-GUS fusion constructs, respectively. In addition, 2.5 kb of the nucleotide sequence upstream from the transcriptional initiation was sequenced.

GUS activity in seedlings from the transformed lines of Arabidopsis thaliana was mainly observed in the root vascular tissue, cotyledons and the tip of the stamen. Both the 1.5P-GUS plants and 3.5P-GUS plants showed GUS activity in root vascular tissue. However, compared to the lines of 3.5P-GUS plants the lines of 1.5P-GUS plants showed stronger activity in root vascular tissue. To investigate the tissue specificity in detail, the roots from 3.5P-GUS plants were embedded in resin. Cross section of the roots showed GUS staining in the cells of central cylinder. No staining was observed in the xylem. The GUS activity was also observed in flowers: at the tip of the filament. For this pattern of expression, no significant difference between the 1.5P-GUS and the 3.5P-GUS plants was detected. Interestingly, GUS activity in stamen was not found in earlier developmental stages. It was visible only after the stage 13 according to Smyth's classification.

In Arabidopsis, dehiscence usually takes place at stage 13, just before anthesis. Cross section of a flower at stage 12 embedded in a resin showed GUS activity in the filament of the stamen which is not dehisced. This result indicated that the expression is induced slightly before the dehiscence.

Tobacco (Nicotiana tabacum) plants were transformed by the leaf disc method with Agrobacterium. 110 lines for the 1.5P-GUS construct and 76 lines for the 3.5P-GUS were obtained. Roots from both lines (13 of 1.5P-GUS line, 5 of 3.5P-GUS line) were stained. As seen in Arabidopsis plants, different GUS activities between the two constructs were also observed in roots of tobacco lines. 1.5P-GUS plants did not show any staining in roots even when they were incubated in ferro/ferri cyanide 3 mM/0.5 mM condition overnight. On the other hand, all the lines of 3.5P-GUS plants showed staining in the vascular strand. For detail analysis, the stained roots were embedded in resin. GUS staining was localized in the phloem of the roots.

Transformed Arabidopsis and tobacco plants expressing 3.5P-GUS showed stronger GUS activity in vascular tissue of roots than the 1.5P-GUS plants. Therefore, it might be concluded that the 3.5 kb promoter has enhancer motifs that are not present in the 1.5 kb promoter-GUS region.

AAP3 promoter-GUS studies revealed that AAP3 is expressed in the vascular tissues of Arabidopsis roots. In addition, in 3.5P-GUS tobacco plants, GUS activity was found in the phloem. It indicates its role in loading of amino acids into the phloem.

The promoter-GUS assay of AAP3 also demonstrated that AAP3 is specifically expressed in the connective tissues of stamen. Interestingly, the expression of the reporter gene was induced just slightly before dehiscence. Dehiscence is generally regarded as a desiccation process. More likely, dehiscence is a more finely regulated process than being just a consequence of desiccation. The regulation may include transcriptional control of some genes. It might be that accumulation of osmotically active compounds, such as sugars, proline and betaine is accelerated and cause water efflux from the anther wall and then triggers the dehiscence. Considering that AAP3 recognizes proline and other compatible solutes, which are known as major osmolytes in plants, it may also play a role in accumulating osmotically active components in connective tissue and triggering the dehiscence. AAP3 may also recover amino acids from dehisced anthers which might not require amino acids any more.

### EXAMPLE 5

### Expression Analysis of AtAAP4 in Arabidopsis by Promoter-GUS Fusions

To study the expression pattern of AAP4, a 2.5 kb promoter region was isolated by polymerase chain reaction on Arabidopsis genomic DNA using Pfu-polymerase (Stratagene, USA). Arabidopsis plants were transformed and analyzed for GUS expression.

68 transformants (2 lines from pGPTV-HPT vector, 66 lines from pGPTV-BAR vector) were obtained. Organs from those transformants were harvested and stained in the ferro/ferri cyanide concentration of 3mM/0.5mM overnight. The GUS staining was mainly observed in vascular tissues of the leaves, anthers, roots and sepals of mature flowers.

The GUS-staining was observed in the vascular tissues of leaves. No GUS activity was detected in vascular tissues of young leaves. In middle-sized leaf, the GUS activity was found in the major veins on the tip of the leaf. Therefore, the expression of the fusion gene seemed to be developmentally upregulated and follows sink-source transition. Sections of the mature leaves revealed the GUS-staining in phloem. Vascular tissues of sepals and petals showed GUS activity. Anther tissues showed strong GUS activity. The GUS activity was already observed in earlier stages of development. In the later stages of flower development, anther tissues did not show GUS staining any more. However, pollen grains released from mature flowers showed weak staining.

To investigate the tissue expressing GUS gene, young flowers with strong staining in anther tissue were embedded in resin and sectioned. The GUS staining was found both in pollen grains and the tapetum tissue. Tapetum tissues seemed to be GUS-active throughout their existence. The GUS-expression in pollen grains seemed to be more complicated. Tetrad microspores showed staining. GUS activity was not observed at the later stage of development. However, pollen grains released from mature anthers showed GUS activity. Root vascular tissue was GUS-active in all three lines tested, the GUS activity was restricted to the vascular tissue.

Arabidopsis plants which express the AAP4-GUS fusion gene showed GUS activity in pollen and tapetum tissue. Promoter analysis of the AAP4 gene revealed that it has a high homology to the minimal pollen-specific regulatory element found in tomato lat52 promoter. As was the case in the lat52 promoter, it may be that some unknown domains of the AAP4 promoter are cooperating with the pollen-specific-element-like sequence to regulate AAP4 expression in pollen. In addition, lat52 promoter-GUS studies showed only a weak expression in the tapetum tissue. Therefore a novel cis-element might exist in the AAP4 promoter which confers the expression in the tapetum tissue.

Arabidopsis plants which express the AAP4 promoter-GUS fusion gene showed GUS activity in the major veins of mature leaves. Microscopic analysis revealed that the GUS staining is restricted to the phloem, indicating AAP4 expression in the phloem. AAP4 might also be functional at the phloem/xylem border, transporting amino acids from the xylem into the phloem.

AAP4 expression was detected in pollen grains and tapetum tissues of the AAP4 promoter-GUS plants. Because of its rapid maturation, pollen might require a large amount of nutritional amino acids. Furthermore, certain amino acids like proline are known to be accumulated in the pollen, probably as osmolytes. Therefore, the developing pollen grains can be the major amino acid sink in the whole plant. Therefore, AAP4 can is an excellent candidate responsible for the amino acid loading into pollen grains.

The tapetum tissues also showed strong GUS activity in AAP4 2.5kb promoter-GUS plants. The tapetum tissue consists of cytosol-rich cells whose primary function is nutritive and it is formed from primary walls by repeated vertical cell division. Therefore, the tapetum tissue may require nutritive amino acids to sustain the development, and AAP4 may be importing amino acids in the tapetum tissues.

1. Regulatory element for the modification of gene expression in transgenic plants, comprising a promoter and a nucleotide sequence, wherein said nucleotide sequence is a nucleotide enhancer and/or repressor sequence comprising a non-coding region of a sucrose transporter gene.

2. Regulatory element according to claim 1, wherein said nucleotide enhancer and/or repressor sequence is derived from Lycopersicon esculentum.

3. Regulatory element according to claim 1 or 2, wherein said nucleotide repressor sequence is derived from intron 1 of the SUT1 gene.

4. Regulatory element according to one of claims 1 to 3, wherein said nucleotide enhancer sequence is derived from intron 2 and/or intron 3 of the SUT1 gene.

5. Regulatory element according to claim 3, wherein said nucleotide repressor sequence comprises the sequence of SEQ ID NO: 1, or the complementary sequence of SEQ ID NO: 1, or hybridizes with the nucleotide sequence of SEQ ID NO: 1.

6. Regulatory element according to claim 4, wherein said nucleotide enhancer sequence comprises the sequence of SEQ ID NO: 2, or the complementary sequence of SEQ ID NO: 2, or hybridizes with the nucleotide sequence of SEQ ID NO: 2.

7. Regulatory element according to claim 4, wherein said nucleotide enhancer sequence comprises the sequence of SEQ ID NO: 3, or the complementary sequence of SEQ ID NO: 3, or hybridizes with the nucleotide sequence of SEQ ID NO: 3.

8. Regulatory element according to one of claims 1 to 7, further comprising a nucleotide sequence of the 3′ untranslated region of the SUT1 gene.

9. Regulatory element according to claim 8, wherein the nucleotide sequence of the 3′ untranslated region comprises the nucleotide sequence of SEQ ID NO: 4, or the complementary nucleotide sequence of SEQ ID NO: 4, or hybridizes with the nucleotide sequence of SEQ ID NO: 4.

10. Regulatory element according to one of claims 1 to 9, wherein said promoter is one of the promoters of the SUT1, SUT2, SUT4, AAP3 or AAP4 genes.

11. Regulatory element according to claim 10, wherein said promoter comprises,
a) the nucleotide sequence of SEQ ID NO: 5, SEQ ID NO: 6, SEQ ID NO: 7, SEQ ID NO: 8, or SEQ ID NO: 9, or
b) a nucleotide sequence complementary to the nucleotide sequence of SEQ ID NO: 5, SEQ ID NO: 6, SEQ ID NO: 7, SEQ ID NO: 8, or SEQ ID NO: 9, or
c) a nucleotide sequence hybridizing with a nucleotide sequence of SEQ ID NO: 5, SEQ ID NO: 6, SEQ ID NO: 7, SEQ ID NO: 8, or SEQ ID NO: 9.

12. Promoter for use in a regulatory element according to one of claims 1 to 11, wherein said promoter is one of the promoters elected from the group of promoters comprising the SUT1, SUT2, SUT4, AAP3 and AAP4 promoter.

13. Promoter according to claim 12 for use in a regulatory element according to one of claims 1 to 11, wherein said promoter comprises
a) the nucleotide sequence of SEQ ID NO: 5, SEQ ID NO: 6, SEQ ID NO: 7, SEQ ID NO: 8, or SEQ ID NO: 9, or
b) a nucleotide sequence complementary to the nucleotide sequence of SEQ ID NO: 5, SEQ ID NO: 6, SEQ ID NO: 7, SEQ ID NO: 8, or SEQ ID NO: 9, or
c) a nucleotide sequence hybridizing with a nucleotide sequence of SEQ ID NO: 5, SEQ ID NO: 6, SEQ ID NO: 7, SEQ ID NO: 8, or SEQ ID NO: 9.

14. Vector or mobile genetic element, comprising a regulatory element according to one of claims 1 to 11.

15. Host cell, comprising a vector according to claim 14.

16. Host cell, wherein the host cell is a bacterial cell, a yeast cell or a plant cell.

17. Plants and parts thereof, transformed with a regulatory element according to one of claims 1 to 11.

18. Transgenic plant according to claim 17, wherein said plant is elected from the group of plants comprising Pinidae, Magnoliidae, Ranunculidae, Caryophyllidae, Rosidae, Asteridae, Aridae, Liliidae, Arecidae, and Commelinidae.

19. Transgenic plant according to claim 17 or 18, wherein said plant is elected from the group of plants comprising sugar beet (Beta vulgaris), sugar cane, Jerusalem artichoke, Arabidopsis, sunflower, tomato, tobacco, corn, barley, oat, rye, rice, potato, rape, cassava, lettuce, spinach, grape, apple, coffee, tea, banana, coconut, palm, pea, bean, pine, poplar, and eucalyptus.

20. Seeds of plants according to one of claims 17 to 19.

21. Use of a nucleotide enhancer sequence as disclosed in claims 1 to 11 to enhance a promoter.

22. Use of a nucleotide repressor sequence as disclosed in claims 1 to 11 to inhibit a promoter.

23. Use of a regulatory element according to one of claims 1 to 11 to enhance or inhibit expression of a plant gene.

24. Use according to claim 23 to enhance or inhibit expression of a SUT1, SUT2, SUT4, AAP3 or AAP4 gene.

25. Use according to one of claims 23 or 24 to enhance carbon dioxide uptake in plants.

26. Use according to one of claims 23 or 24 to enhance plant growth.

27. Use according to one of claims 23 or 24 to enhance sucrose phloem loading capacity in plants.

28. Use according to one of claims 23 or 24 to increase sugar content in plant tissues or organs.

29. Use according to one of claims 23 or 24 to increase amino acid and/or protein content in plant tissues or organs.

30. Use according to one of claims 23 or 24 to increase oil content in plant tissues or organs.

31. Process for the production of a transgenic plant, especially of the genus Beta vulgaris, comprising the steps of transforming a plant cell and regenerating a plant therefrom, wherein said plant cell is transformed with a regulatory element according to one of claims 1 to 11.

32. Process according to claim 31 for the production of a transgenic plant, wherein at least one of the SUT1, SUT2, SUT4, AAP3 or AAP4 genes is expressed under the control of a regulatory element according to one of claims 1 to 11.


SEQUENCE LISTING (9 sequences)

SEQ ID NO: 1 - DNA, 522 bp, Lycopersicon esculentum
gtaattttcaaatcgaaattcgaaaattaaaattgtgtttgaacatatatttaatcgaattattactgttttttttttttaactagctagttgctatattttttatataattaaaatcatataaagactacatgcaatacaaaattccagctagataaatgtaaaaaaataatataattttaggtacaaacaaaaaaaaaatagttctaaatatttgcttaaaaaataattaatggaatttcctcaataattattcttttctaataacatgttttttaaaagatcaacaactttattctgttttttttaataaaaaattataaaaggtcacaagttgtaatttatttctagtttcacttagttatatataaaatttgattgtttcgatttttgatttaattataagctcatgtgttactagattaataaaaaaaaagtcacatgcagtgattacagtgttaaaatcaatcaacttttattactaatatttatgcgtcataattaaattgtttttattttcag

SEQ ID NO: 2 - DNA, 980 bp, Lycopersicon esculentum
gtgagtttctttccatttattattacgataaataaaattttagatagtcaattaactgaattaataaaatatttttttacaaaaaatttaatatttatatttagtatgtgtgaacatatattacaggtgggttgactcataagtccaaatatgacaaagagatttattatgattttttaagaattttggtttttttaggttggtggaaaatttatattatatggataagcaaagtgtgaaaatattgaccaaatatgactataacaaacacaaattatttgttagttatatacatgacatcctcttcatatataggcatgtaaaattaattaaatggttttctttgtctttgactatatttgaaagtaaacattatatatcatttttgaaattgaaattggatatttagttatatcatttttaatagcaattaggatgaacattagccacatgtatgttttcaatggataatcaaatttttttttgaagaaaatttaaagggagtataggttatggcttttaccataaatagatcatgatatgtatgtgggcttgtaaacggttgtaagtcacacacataaatgtagaaggtgtgagaagctttttattaatgcatcacatgactttttagttttgttaatttctagttatttaaattaataattattttatgaataaataattttaataaacatgacaattaaaaagaatcgactattatttctctttatcctaacataaggattaggagaatcttattactttttgattaggataataatacaagtttctatgcaaaatcatcttttattattattattatggcaaaagcatatatctctcttcttcttcctttaaattattcttttaagatagaattatttcacttttgatgtttagattttccctttttatcttttttggcaaattgatcatgtttttttactaatatatgaaataaaaataattttattttacag

SEQ ID NO: 3 - DNA, 684 bp, Lycopersicon esculentum
gtactatttaatttttaattcctcaatcatggataacgtacttaattcttctttttaattatcttacctttcataaattaatgcaccatatatttaaacacaaacgtgtatctaaatagtgaaaaatatataaatgctagtctattacgtgtatatactgttatagatcgaatttatgtacgaaacattaccaatcaaagcaaatgtagtatcgttttcaaactattgacagtcttgtagttaactaattaaatttagatctccatcctacaacgcaagttgtaaaaacacctcgtaaatccggacacatgttgtaggattaggggtagatgttttcgaggctattaatagtgtagagataaaagttttggagtgttttcaatacatctgtctcgttagaaaaaggtcgaggcccacgttaaccggccattgtcacctagacccctacaataaaaaaagaaaaagaagaacaacgtataaataggggaagatcataactgaaggaatttgaaaatgacagaactagaactacattaatgagatatgaccacaagatataatcttatgactatacttcttattattcacattaggataaactaacatactattattgatattaaattagttttaaatacacaaggacctaactataactacttgttttttttttttatgatatag

SEQ ID NO: 4 - DNA, 1231 bp, Lycopersicon esculentum
aaaaaattacaaaagacgaggaagaagaagaaggagaagaaacaaactttttttttatatattagtacttctcttttgtaaacttttagaaaacaaacataacatggaggctatcatgtccatgtatcttcctttttttttttcttttcataaagctctttagtggaagaagaattagaggaagtttcctttttaatttcttccaaacaaatggggtatgtgtagttgttttcaatttgtgtgtaacaaagaaagttgtccagtattaacatgtctgggcaaatgctttgtacccttttactatttttatttttatttttggtaattttcttcttggaaggtaggaaaaaaaaaagaagtggttggaggtggaagtggggtactttaatttgggttggtaagaaggggagatttatgaaaaactttgtacttaaagtggaatcaaaattgatctatctatatatctatcaatgttttctatttttttgtttcttgtttcaaccattatttgaactttttggtggattgatagcactattcttaccttatttagtggtatgcatcttctagcacttcccaaactttgaagtattaattaataacaacattaatttgtaatatacttgtattatattgagtggtggagctacgatactaaagttagtctactatgctctcaaagagttgttaccagtgagatttgaacacataaatactttatatttgtgctaataacctggtttggaagcaacaaatcacaggagcgtagtttcttttgtgcttcggggaagaggctgctcagctcctataaaagctaattttgattggtatcattcaaatgcgaatataggcctagtcgcattgctcttttaagacgcttgaaacaattgagttacatcatattcttatgtcaagaatgtcccaaatatgaaattactttagttattgtattacttgtatataatgtataatttttcgacaaagggtctataattgaacatcagtgcaactacgcccctaataactattggaaatttgtgtttgttttaccatcctaaatactagtgcactaattggaataagttatatataaatattttggaataatattttctattcattacaaaattaaaaacaaaaaataacaaggttttttaaaacacttatttgaacaaaatttcatgcgtaaattgaaagggaattttactggagataaataggatttgggagt

SEQ ID NO: 5 - DNA, 2354 bp, Lycopersicon esculentum
gaattcttgtgtatcatatgtgagatataaaagaaaatgaatttttttttatttaaaaaaaaaaacgtgtcaatacgatctttttctagctagagcaccattttcaattaattttacaaaaatataaaaaaataaaatataaaatgaaagtagtaaagaatcagagttgtctttatgttgagacacaaaaatttatgaaattttttaatacataacataaaaggggaagaaaagctgcaacagtgaatttaatgataataaaacttactgtatgtttttctggtcctatatttggataagtctccacttctactttttccctttaattaattcttgactagagagtttaaatttgtaaaattataactatgaattgacttttgaaagttactttttctttttcaatttattacttattaatatgaactcttgtttgttttgttaaaacgtaattacttatgttagagaaaatattttatatgaatgtggtactttttatatgactttaaatttaattggttttgatgtaaatgtcaactatcacaattcatatgtaaaaaaattttaaaaaaaatattatttatcataagaaaatttattttggatcctattatatcttaattgttatcgttcacttatatctttaactatattaaaaataaggatgaattacataaatttactagtttagtgttgattatttagatacaagtttatttataatattacgtatttactaaagtttggtgtgtttgatacatgtgtctaggcaaatatgtggtcgaaatttaagtataagttgttctagatataatgcatccagttggtgaattcgcatgtaccatgataatatatagacaaatcatgctcgcctcctttccatctcattcatcgctcttttatcttgctcgcatatatgaagtgattcgcatgtgtatgatatacacacgtaaaaagatctcgcttgcatctctcgttttctcacatacatatacatatatatatatatatataaaacgatcttgcttgcatctctcgttttctcgctccctcccttcctatatctactagcaaaaaatatgtatgtagatatatttgatttgaaagttgggggtgaatttattaagactattgggataaaatgctattataaggagaataaataaatatgattaataaacattatgcatttagatagtttcgcgtaaaaataaatatatcttgttatttatttaatttcaaaaattaaaaaataattcagaacgcttttgttttcctcactcctcaccgtccatcaggacacttctactttcctcaatcctcaccgtcctcggcttgcttacgtagtaaaaaataaagaaattgtctatgacagatggaatacgagatctaggtaagtctttctttttttgtcaactttaatgctacgtagactttattttattcttatttacccttataataaatattagttataaacaaatttgatatctcaattggttgactacctgaacatttatcttgttagcaacgattcaatttcttattttataatttttcgtctcatttctcatttcccgtgttcccattatacttgcaataattaaatataaaatgtaataaacttaatagattttttcatataattttttaaaaccgatataatattatttgtggaagcttgaaagaatgataaatcaaggtgatatatgtacatactagatacatttatttttccaaacatgtgaaaccactaccaatctagcattcaatagaatttcctaacaataaacaatatttgaaataacgaaaaaagacaaaaactttattaaatgatagaagcgtttcgcttttttagaattttttctacttttattacccaaactaaaatttcaaaatctacttttcccatttaaataatttcattcttaaatccttttatttatttttttaatataactgggtctggggtggcaacagtgggggctggttcttaaaaacctctgtggccggtcactctatcgttctcaaatttcactcgtaaaaatttcattgaatatattattagttcaatacattttaaatttgataaaagatcaaatcagataggtaaattgaaatggatgaggtaattaaattatttatgaaaaatacaaaaaaatttattacttttgactaaataaataaaaaaaggatattattctaccacatgcatgactattgcctccaaaaaaacactataaatacccccttccattatccccttttcttataacattcaaaacccaaaattaaaacagagcaattttagtttcttagaagaaaaaaaaatggagaatggtacacccgggtac

SEQ ID NO: 6 - DNA, 3100 bp, Arabidopsis thaliana
cctagtctccgacttaagactcgagatccggtctttctggtggcgcgtgaggtcggccacggagacctcggtgagtttaaacacaagagaaggcttgaaatcgccgacccataaaataagtttagcgtaggagttaaaccacggtgggcagaagaaagtgaaaacgttgtccccggcgagggaaacggaggcgaatttctcttggtagtattgaagacagtgagatagaaactgggccacgagagattgttgttcttcgagagagatacgattagtttcatccaaggaagaagcacacataagctgttcaacgaaatacctgtgacggattaaccaaccatcaacgaagatagtgaagctttgagagctgctagtgtttggcatgtttcttatgtttggagtactcactaagtaagtatggactgtgagtgtacgttgtgcaagaatgtttatatactactacattgggcttgaccaagaaaaatcttttaggaattaatttacgatatgtcggtctatcgacgatgtgtcggcgggcgaaatggatttttgtgacttttgtcttgtcggagtgaagtgtaatcagagcagcgtcttatcatcatttgctgttgttattcaactctggttttagaatcaagttgggttaaacaatagtataaaattttaatatggatgaaaataaaatataaattatccttggtaattattgattataacctatagaatcgacaaaaagaaagactataacctagagaatttttttttttttttaaaacaagtgttcattaaaaatctttaaaatatttagtctgttttataattaaaatgtttatatctgaaattgtatctatcactactagttccagttggaattatatatctgtcagggtcgcatctcgatattatggaatgacctaaaataacatctgaagaggaaaccgacacatgttggtaaaataagaaggcacacgtcatgtaaagaagatgacatatgtcaggaaaggaacatgtccttacaaaatagaaaccaaataaaccaacagttcataaatgctccagttatgccgcgagccatcttttcttctttactaagaaaatttctacattattttaagatttttcttttccatgtagttgtctcatacttccttcgaaaagatctttctctaacttcaaccttggggatcgagcaaccatatttcaatcaacagattctttcatcactctaataacggacaaaaaaagaaaagatgtcaaaaaaaaaaaaattggaatatgcacgttgatactataagttcatttaaatcagtcgttagagtaaaccttattgagagttcaaattcatttaaaaacctgatgagataattatataatttcttataagatttttttttcttgtaagaatttaacgaatagaatttttctctctattacttaaaattcctcctcgtcaaaaattgcaaatgcttggcagttttaccattttctcagaactaccgtatgagaaataaagtacgtattaaaaaacaatacaagctttaaagataaaagaggcaacaaatttttttgacaaaaaaattagatgaaaagaagtgacgacaaataactgagaaaacacgttttgcaaaactaaaaaaaaaaaaaaaaagagagagttggaaaatgtttggagctggtgggcattagttggctatgcaacgcacggacgcggatacttttcaactcgttgcttacgcggccacgttttcgtcacaaaccgtgtttcttttgcggccttttaagttttaatcaaacatttttgttttcatttgaatcttttttttaaggcggttatacgaataaaatcattaaaaggagaaacgggccttcatgtttaggtttttgttcttcctcatttgggttatttagcttattggttcagcccacaagagtttccttcaatggatagggccttcagttgcatgccatcatcctttcttgtttctctataagccatttttgcaatgagaatttctcaatttcttatttttgcaataaaatcattttgccttagatgatggtggaatcatccgtttttctttattttgacccggtgagtaattgaacgcaaaaaatagtccaattattcaaaaccttacaatttcatatcgccataataaacctaaacatgattcgagtgagcaactacataaaaaggggaacgaaatgttggtacaaaactgaattccaacccaatcaagtgcatactaaactattgagcaaaaacaaagataaaaatcagttcacctaccctaattaaaacatgattaggcctaaaagactataagtaaagataaaaaagatgccattttagagtacgttttggtaaattggcgccatcgtgttatacgtttaaaccgtataacctgaatcatgcagcacgtgtacggctcacattggcccaagcttgattaacaaacaataacgtcattttcgtatttcaacaattttgatcgctcaatcctttcgacctaatttccatattaagtaatttattagttaaaacaacggactcaaccaaattacatattaagagaatatttgaaaattgtctatgtagatgaacatgtatttgaaaattgcgtaagcacactctttctctctctcttttttttttttttttttccccctcttctttctttgaataaagatgacatttttttctgttccaaacacaaaactgcatattgatagtgttattgaatactcaacgtctcttcttccagcttacgtaagtggaacttactttttgccccgtgatccaacagaaaatgacgaatgctccctcaaaatcatttcgtttcaacaaaaatatgtattttatccatagagaaacaacaaaccttacactatataccctcttttaacgtgcccacttttcattatttacacaatttccccaacaaatttaaaacaatttacaaaaagcactcaactttcctaaagtagattcctttagtccaatggaattcttacaacatcttcttcttcgccacctttttctctccactttaagctttttcactctctctacgccacgacgactgtgaagcgagaa

SEQ ID NO: 7 - DNA, 2441 bp, Arabidopsis thaliana
taatgattaatgaccattataatttttctttatcaactttaatggagttattatgtgaaacagtgtaagccccattcttatgtatatatgtaaatatcacattataaattatgatctatcgacagagaacgacaaaattgccaaccaatcactaagttagaaaattaaatttattgtaataacgcatctctttttttttttttaaacatatagtttttatattattaatatttgaatttgtcgtttgagaaatgacgaaggagttattttcaggcgtaagcatttgacgtgctcgacgttaaggagatgacgtgttaagttatggtacatatgtttttttctcttttatttgggtcatagccgcgttcaacacgtgctttttgtcaaatgtaattacatgattaaactaaaaaaacttattactgtttttcaatcaacttattaaccttgatcttttttataacaaaataaaaataaaaattggcacatgatcaaatcatcaatctcaaataagataaaaccaaaggtgtaataaggttcatttttttgtggagaaaccgagtcaatgctaccaaattgcctatataggcttgaattttatgtaaatgcttttttgaatggaataatccgattttttattttattttttcttatgtaaaattataacatggtatagataaaaaatttgttttgtaataagtgatatttggtctaacggcgcgtatgtatagttcttttacaaaagtgagtaatagtggttttctggttcaaatactattgagaatggtatcagttttgatggacaacaagtgtatgagactcggctataacttctcaattaaccattaaccattaaagaatacaaaatttggattttcgactatactttggttcggatacatcatacatgcatgcacaccataacaactaaactcaaaaggatttgtttttgaacttttgatgataagaaaaagaaaaaaaagatgggacataagagtcatgtacacgtggataaggaaaggagaagagagggacaagggaagagatggagaaagagagacgtaagtattaattcaattgtgtgccctattagctttcattttccacaagcaagaataatattaataattatgtatcatctttgcatttttgtaccaatacatgatacatttgttaggcacacgtctttttcctttttcctcctttttatttctaactttcttccttttattcgtttaatccggctctatcacgttaacacacatatatgttatatcatctttttacaaacatgtttacattagttattagaaaatacatatacttgtaatagtgacattggtaagaagaactctatcaccttcacaatcacacacatatatgttatataatctttttacaaacatgtttacattagttattagaaaatacatatacttgtaaatagtgacattggtaagaagaaaaaaacactattaaatagtgaaaaaatgtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttatccttttttctttaataatatgcaattgtgagagtttggatttgaatggtagcattagaagcaaacttgaaccaaacatatttcatgaagtcaaacttgaaccaatgtaatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacaccgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcatccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggacg

SEQ ID NO: 8 - DNA, 2192 bp, Arabidopsis thaliana
gatcttaacttttatttaaatactactattacagatcattatctaaatagaaacctaactgataatagtaagcaacgaatccgatgcctcctctttctctaaatgatcgaatgccgataaatccgccggccgtcctgaaaagtgaaaagggagaatgtaagtgttaggaggatatagtactcatggcgatgtatactcttacaaagataataaaaataataatgaagataactcttactaagggatcaagttgaagaaagtgcagttgctatgatgaaagggcggtttctcattcatttttttaattcgttaatcgacattgaggatggctgtgtgtgtattgtgtgcgcttaaatcaagtggatgatcttaaccaaagaaccatattctttttcatgatctcctagatctttgttattacttttaattatcttttcaatgtaaacatggaaaaaaaaaaaaactggtacattttgtatgcatttttgcggttataaactttggaattgtaaagaatgaactagattttaacctgctgacaccgagacaatttatttttttaaattagtatatataaaattttgcaaattatatctatctataaaatatttttattttatagtttataattattattaagtaacatccttctaaacccgttccaccaaactcgtcctgtaaaaaaaaaccgcggtaccccgtaatttaaatttataattaaaataaatatttacctagggattgcaattgtgtttagtttgtcaaacaaaattttgtttaaatagttgtcgaaatctagttcaaattttggagacataagatttagtgaatagataagaaatgtatttagttgcgttgctcattttaagggattgcaattgtcttttgggttgtcaaacattttttgttttaaatagttgtcgaaatctattttaaattttggaaaaatctgcataatatatgggattggaatatatttggaatgttttttcatttgttaatcaatttattgctgaaagaaacaataattaaaagccaatgacagccattgtaaataagttccaacttcaggatttatttcacaaaatttcacaaaatgactgcaacaatgtatatataaattctatttttatgaatataattccctttctatttggagactattttctttgaatgatgacttgtctgaatacaaatctcctcgtaagtccttaaaggatatttggtttcccacgaatcaagccaatacaagtttgtgtatgtcatttatggtttaaatgagtttagttggcaaagtaatcaaaatatttcatttcgagaagaacatatagcttacacgcctcataagttagttacctacagaaacttatatggtggggtgatatactttcggattctaggtaagatatagtatatgtgcgttactattgctcatactataaaaatcaccttttggttttgagttatcattttgaaatctacacatgattgcgtgtactcttatttaaagtcggcatagatttctccatatatatgtcatgattccatgaatgttcttttaacatcttaatgcgattttacattaaaatattcaagaaaaaaccaagctgttttctaaataaatttagtttgtccacggttaataaaatttaaaattagcgaaatacgtaaataatcaaatgatataaccaaaatgcctattatttttaaaacaacattttattttctctaatctcataaaatactctaataaaatctagttgtaagtgaaaataacactaagcttcaaacacacacgtacatgaatgtttgtttttttctttcaaaaaagctgaacaatgacattttaagtccaaatgaaaacgctggtatcatcttggacttccaattttccattgcccatattttctcttttcttttgtttttatccaattggtatcgaatcattcatataaaattataaatcatgaagtcaaatttttcaatagtaacacttgagtaatagtggtcctaccaactttttaccaagacagatatattttttttaatacactttttaccttttactgtgtttgcctcaaatgagtgtttttaccagtttctgtgtatataaatctgcactccactctcagtttatcttcatccacaagacagttctttaacaaagtcattaaacatttcccttaccttct

SEQ ID NO: 9 - DNA, 2694 bp, Arabidopsis thaliana
gagattgagatgggacctctgcgaatgggtgggcttgtagcttcctcgggtacaaaggtgttgactatccgcaaaccgggtgttcgagtggaccaagactggaccgtaaagattctgtgattgaacggtttgaaccagtttcactcatggccgttagagtggccgaaatctgcttttccggcgacggaatatcacactttttaatatatgtttggagatttagacttaaatagttgtaagagctaacagtttgaaagtcactttgcattgttgtttatcttcatataaatgagtttagattttgataatttcagaattcgtggaatcataattaacaattttgatagggaaaaataatttgttttttttagtcagagggtcaaataatcttaccttccatcgttgcctaattattgtcaaatccttttactttttacctaatcaacccatgaaaaatcttgaattacacgttttaagagtcatgaagaaaaatcaatctgattctgttggctagatttgttttttttatactgcttagtgtttactgttatagaaagggttctaagtatgcaaagtttgccctaattagataatgttagatccgtgtaggtgaaacaagaagccaaaattctgtgtaaaaagagtcacgtaactacctaactcttgtaggtctaaagaccatcttcactttaaattagaacctcatgtttcccaaacaatatattccgttcatgaatctcgtttcgaattaaaactaaaatattacacattttagaatgtaagaaaaacacactcaaaattaagaatcttggaattgaaaacgagattttgaatgtgttcaaagatactctttagaaattataatggtagataatagcacttgcatgacaaattcgattctctcttctcatcatgcatttttgctttacattttgttcctttaaaccaagaatattttgactcacctaaaagataaaccaaacatatctgaaatctacaattgacaattaacttctctttttcttttctcttattcttactgaaataaaaatagtatgcaactggtctttataaatttcagttttatatatttaaaaaaaatcttctataccaagtttaccaactcatggtagtccaatgattaaaaaataaatcattatggggttaggacttaggagatcgaattcgaatcatttcaaattttcaatgcgatttcaatcattgtttttcactgttaacactttgaaatatctaagtgtatctaaaaatttcaagccttagagattaatttggacttcgatccggaattctgcgacgactatcaaacaaattttgttttaaccgaaatatagattatttgcatctctgattatcaaaccattcattctttttattattattcagtttactataaagtatatgtccctccctttatgaaccgtatcaaatactatacatagtatgcatgttcgaagaaatttccaacatactaatttgaaatcaacgaacatatcaaattataatgcgttgatgcagaatatatgctttctatatatataacttaaatacaaaaacctaaattttccatatgccttcgtaatttagtatataaatattcattcattcttaatacagcctaggactcggtagcattttgaagtaggaacgaattttgttaattaagatacatgagtgattagatcgaatccaaaaggtatggaggcgtagaacaagaatttggttctctttcattttacgtatctttcactaaagaatagagagaaaacaaaaccatgcaaaaacctctcaaaaattatatattgaacgcttagacaaagagccaaatttattcgctttttgatgtttacatcagtatttgtttctagctagttcatattatttaaactccataatcatttgaaagacataaaatacagatgtttaagatctgtggatgtttatataggatcttcgaattcggataatgaaatcatttatacactattaagtattaactttgtagaggacttaagtattcgaaatcttgattcaggttgtcaatcaagaaccgtaatgtttgatctatagagccatgggattaatggatgctaaaagaataaaacactcgagaagaagagtacacgtaaaatcactcacacacaaagaatgagagatgggagaagaatactacaaagtgtttgttttttttaaatcggctcatatataaatttgggcgatactctactacccacattattcttttaaacaatgatataaatgtgtatggcctatgtagccgaactttttacatatctgacctgtatatatataataattatttatcaaagaaattagagaaagattcttccacttttgatggcttataaaagatgatgacccaaaacactcaaaatgcagcagcaactgctctctcaccacccatctcctcatttctcttttgcatttctttctttttttctgcattgcattcttttgaggggtttaattttctgcatagctttgtctaatctcttagagctcaataagagaaggtactataactgatctctccctctttcaagttttttttttgtttggtttatagaaaactataaccgccattatccgttagttttaaccgttttttgaaactagtgaatcctcataactttggtttctcactaaaaacagatg